(19) United States
(12) Patent Application Publication
(10) Pub. No.: US 2018/0177522 A1
(43) Pub. Date: Jun. 28, 2018

(54) METHOD AND DEVICE FOR MONITORING A TREATMENT OF THE HUMAN OR ANIMAL BODY

(71) Applicant: SIGNUS MEDIZINTECHNIK GMBH, Alzenau (DE)

(72) Inventor: AZAD KHEDER, KÖLN (DE)

(21) Appl. No.: 15/324,822

(22) PCT Filed: Jul. 9, 2015
(86) PCT No.: PCT/US2015/001396

§ 371 Date: Jan. 9, 2017

(30) Foreign Application Priority Data
Jul. 9, 2014 (DE) 10 2014 010 153.3
May 28, 2015 (DE) 10 2015 006 914.4

(51) Int. Cl. A61B 17/34; A61B 17/3211

(52) U.S. Cl. CPC A61B 17/3403; A61B 2017/00039; A61B 17/3211

(57) ABSTRACT

Process for monitoring a treatment on the human or animal body, in which, in the area of a nerve cord, current pulses of a specified pulse width are applied in the body and in the area of a muscle that is in communication with the nerve cord, voltage signals triggered by the current pulses are measured, in a preparatory step at least one criterion of voltage signals which are still measurable in response to current pulses being determined and during the treatment, the amplitude of the current pulses being adjusted in such a way that the voltage signals received in response thereto approximately satisfy the criterion.

### CROSS-REFERENCE TO RELATED APPLICATIONS

This is a national phase application from PCT Patent Application Serial No. PCT/EP2015/001396, filed on Jul. 9, 2015, which claims priority to German Patent Application Serial No. 10 2015 006 914.4 filed on May 28, 2015 and German Patent Application Serial No. 10 2014 010 153.3 filed on Jul. 9, 2014, each of which is incorporated herein by reference in its entirety.

### STATEMENT REGARDING FEDERALLY SPONSORED RESEARCH OR DEVELOPMENT

Not applicable.

### BACKGROUND OF THE INVENTION

The invention relates to a process for monitoring a treatment on the human or animal body, in which, in the area of a nerve cord, electric current pulses of a specified pulse width and a specified frequency are applied in the body and measured by the electrical voltage signals triggered by the current pulses in the area of a muscle that is in communication with the nerve cord.

During surgery on the human or animal body, it is necessary to prevent damaging the nervous system more than absolutely needed. This is particularly important during surgery in the area of the spinal column. In this area, numerous nerve cords emerge from the spinal canal to innervate muscles, which must be protected during surgery. It is possible to exploit for this purpose the circumstance that, via the nerve cords, current pulses are transmitted to the muscles to be innervated and that, in the area of the innervated muscles, a voltage response to the current pulses can be measured. Corresponding current pulses can also be applied in the body using an external apparatus, the amplitude of the current pulses which is required for triggering a measurable voltage signal with a known current pulse width at the innervated muscle being a measure of the distance between the location of the current pulse application and the nerve. This knowledge is utilized, for instance, in processes for surgical treatment monitoring described in U.S. Pat. No. 8,050,769 B2. In this known process, during the surgical treatment, a current pulse sequence with pulses of increasing amplitude is continuously applied in the area of the wound or in the operating field. When the current intensity required for generating a specified voltage response of approx. 100 μV in the area of the muscle decreases to below a specified threshold value of 10 mA, the surgeon is alerted that he is approaching a nerve cord with the instrument by means of which he is applying the current pulses in the human body. When the current intensity required for generating the critical voltage of approx. 100 μV decreases to below 4 mA, an additional warning signal is triggered that informs the surgeon that there is now an immediate risk of damage to nerves.

Although this known process allows a helpful warning for the surgeon to take place, it has been found that monitoring can simultaneously lead to an impediment to the operation. In view of these problems in the prior art, the invention is based on the object of allowing unimpeded monitoring of a treatment on the human or animal body.

Procedurally, the object is achieved by an enhancement of the known processes, which is essentially characterized in that, in a preparatory step, a criterion of voltage signals that are still measurable in response to current pulses is determined and that during treatment the amplitude of the current pulses is adjusted in such a way that voltage signals received in response thereto approximately satisfy the criterion.

During this process, within the scope of the invention, electrodes for signal derivation, such as those used in electromyography, can be used. Both the use of bipolar needle electrodes and the use of monopolar electrodes is possible. Moreover, surface electrode arrangements, such as those described in U.S. Pat. No. 8,050,769 B2, can be used. Particularly preferred, within the scope of the invention, fine-wire electrodes will be used.

Signal derivation and signal processing, including signal filtering, can take place just like in known electromyography processes. Signal derivation and signal processing using this process are, for instance, described in “The ABC of EMG, A Practical Introduction to Kinesiological Electromyography, Peter Konrad, Version 1.0, September 2005”. The disclosure content of this publication is hereby incorporated into the description herein by express reference as far as signal derivation and signal processing are concerned.

As described in the aforementioned publication, during signal propagation along the muscle fiber, a depolarization-repolarization cycle occurs. It causes a depolarization wave, which is propagated along the muscle fiber. If, during electromyography, a signal is measured as a potential difference between two electrodes that are arranged along the muscle fiber, a bipolar signal is obtained, which is a function of the distance between the electrodes and the potentials of all the muscle fibers excited within the detectable motor unit. Such signals are examples of voltage signals triggered by current pulses in accordance with the invention herein.

### BRIEF SUMMARY OF THE INVENTION

The invention is based on the realization that the problems occurring in the prior art are essentially due to the fact that the current pulses used for monitoring are regularly above the depolarization threshold so that the current pulses of a specified amplitude leading to a voltage signal cause a muscle contraction. This obstructs the operation. In the known processes, a high detection threshold is permanently set for the voltage signals, in order to assure that a reproducible voltage response can be ascertained.

In contrast thereto, in processes according to the invention, for each operation and for each patient, a criterion, such as a detection threshold, which is regularly below the depolarization level, so that current pulses used for monitoring, whose amplitude is always adjusted in such a way that the voltage signal approximately corresponds to the criterion, in particular the detection threshold, do not cause any muscle contraction. As a result, trouble-free monitoring of the operation or treatment of the human or animal body can be assured. In doing so, the criterion can also be a temporal development of the voltage signal, such as a rise or a fall of the voltage signal that is measurable in response to the current pulse. The temporal development of the voltage signals can be measured with sufficient accuracy if the voltage signals are measured at a frequency or the signal development is detected or scanned at a frequency, which is greater than the frequency of the current pulses, in particular at least ten times the current pulse frequency.

Additionally or alternatively, the criterion to be satisfied by the voltage signals can be a detection level of voltage signals that are still measurable in response to current pulses.

In processes according to the invention, as a function of the circumstances of the individual case, the detection level is between 50 μV and 150 μV. As a function of the distance between the location, at which the current pulses are applied, this detection level can be generated by the nerve supplying the muscle equipped with the measurement arrangement, using current pulses, which, as rectangular pulses, have an amplitude of 2 mA to 100 mA and whose pulse width is between 10 μs and 300 μs. In preferred processes, the pulse width is between 150 μs and 250 μs, particularly preferred approx. 200 μs. The pulses will preferably be applied at a frequency, at which it is possible to assure that, between the individual current pulse sequences, a period of time elapses, within which the response to the current pulses completely decays.

In processes according to the invention, the detection level is usually less than 100 μV. At this level, the depolarization threshold is not reached. No muscle contraction takes place.

By means of empirical studies, a relationship between the distance between the location of the current pulse application and the nerve, on the one hand, and the current intensity of the current pulses, which bring about a voltage signal at the detection level, on the other hand, can be determined. It is then possible to determine that a dangerous approach of the instrument, by means of which the current pulses are applied, is taking place on the nerve if, for obtaining the detection level, current pulses with an amplitude are applied, which is below a specified threshold value. Therefore, a preferred embodiment of the invention provides for a first warning signal to be triggered when the amplitude of the current pulses required for obtaining the detection level decreases to below a first specified threshold, potentially determined by empirical processes. As the instrument which applies the current pulses comes closer to the nerve, the amplitude of the current pulses required for obtaining the detection level decreases further. Damages to nerve paths can be safely avoided while, at the same time, preventing any unnecessary impairment of the surgeon if a second warning signal is triggered as soon as the amplitude of the current pulse required for obtaining the detection level decreases to below a second specified threshold value, the second specified threshold value being smaller than the first specified threshold value.

When using such monitoring processes, the surgeon can even operate in the range between the first threshold value and the second threshold value without any problem, provided he exercises particular care there.

For obtaining the detection level, expediently a mean of the voltage signals triggered by the current pulses and the average deviation of the voltage signals from the mean will be determined, the detection level being defined as the level, at which the average deviation decreases to below a specified threshold value. The threshold value can be established as 50 percent of the detection level, preferably as 30 percent of the detection level. Hence, if the detection level is approx. 80 μV, it can be defined as a detection level as soon as the average deviation from it is less than 40 μV.

In a particularly preferred embodiment of the invention, for obtaining the detection level, the spontaneous activity of the muscle is measured without any stimulation by current pulses, and the detection level is set to a value, which exceeds the level of spontaneous activity, usually approx. 30 μV to 60 μV, by a specified level, preferably 30 μV to 50 μV.

When using processes according to the invention, it is possible to obtain a particularly low detection level if the voltage signals are amplified by a carrier frequency amplifier whose carrier frequency corresponds to the frequency of the current pulses. In a carrier frequency amplifier or lock-in amplifier or phase-sensitive rectifier, the measurement signal is multiplied by a signal of a specified frequency and is subsequently integrated into a low pass. The cross correlation between the measurement signal and the reference signal is thereby determined for a fixed phase shift. The cross correlation for signals of different frequencies is approximately zero. The carrier frequency amplifier, therefore, does not provide any output signal if the frequency of the reference signal differs from that of the measurement signal. Only for equal frequencies does the cross correlation provide a finite value and the carrier frequency amplifier, therefore, a finite output signal. Hence, by selecting the appropriate frequency, in this case the frequency of the current pulses, the corresponding component can be filtered out of the measurement signal. As a result, the signal-to-noise ratio of the measurement signal can be significantly improved. In processes according to the invention, it is, therefore, possible to determine a reproducible detection level even at very low voltage signals.

According to an additional aspect of the invention, which is considered to be patentable autonomously and independent of the embodiments of the invention explained above, a time interval between the application of the current pulses and the measurement of the voltage signals is set. This can, for instance, be done, using a so-called boxcar integrator.

This aspect of the invention is based on the concept that between the application of the current pulses and the detection of voltage signals in response thereto, a transmission period corresponding to the transmission distance between the location of the application of the current pulses and the location of the voltage detection passes and the detection sensitivity in detecting the voltage signals can be increased if a measurement takes place only during a specified time interval after application of the current pulses, a situation, during which the time interval between the application of the current pulses and the detection of the voltage signals corresponds to the transmission time and the duration of the detection interval can correspond approximately to the temporal length of the voltage signals.

Using such a process, the detection sensitivity in detecting the voltage signals generated in response to the current pulses can be increased to the extent that reliable voltage signal detection before reaching the depolarization threshold can still take place even if a detection level such as 30 μV to 150 μV, preferably 30 μV to 50 μV, is firmly specified.

It has, however, been found to be particularly preferred if, even in this embodiment, a detection level is determined in a preparatory step and a response to the application of current pulses is only accepted when the detection level has been reached. For this purpose, in this embodiment of the invention, the spontaneous activity of the muscle can also be determined in a preparatory step. The detection level can then be set to a value which exceeds the level of the spontaneous activity by a specified rate.

Particularly in the last-described embodiment of the invention, it has proven to be expedient if, in the monitoring process, a first pulse sequence of successive current pulses with a first current pulse having an amplitude of the first specified threshold value, a second current pulse having a current amplitude of the second specified threshold value and at least a third current pulse having an amplitude that exceeds the first threshold value is applied, a release signal being generated when none of the current pulses results in a voltage signal which is measurable in response thereto, the first pulse sequence being repeated when the release signal is to be generated.

Using this embodiment of the invention, a safe continuation of the operation can already be signaled when current pulse sequences having only three current pulses are generated, for the purpose of securing the release, the third current pulse being able to have even an amplitude which is above the first threshold value.

According to an additional embodiment of the invention, in order to accelerate the detection of a surgery situation, in which increased attention is required, it has proven to be expedient if a second pulse sequence is generated, in which alternately a first current pulse having an amplitude of the first specified threshold value and a second current pulse having an amplitude of the second specified threshold value is generated, the first warning signal being generated and the second pulse sequence being repeated if a voltage signal is measurable only in response to the first current pulse. Hence, in this embodiment of the invention, a surgery situation, in which increased attention is required, is demonstrated using current pulse sequences each of which has only two current pulses.

According to an additional preferred embodiment of the invention, a particularly critical surgery situation can be monitored at a high speed if a third pulse sequence in response to the detection of a voltage signal is generated as a reaction to a current pulse having an amplitude of the second threshold value, at which alternately current pulses of different amplitudes, each of them below the second threshold value, are generated, a current pulse having an amplitude corresponding to the second threshold value not being generated again when in response to no current pulse of the third pulse sequence, a voltage signal can be detected.

This procedure allows particularly dynamic monitoring of a critical surgery situation using current pulse sequences each of which has only two current pulses. If after the termination of the third pulse sequence, hence when in response to no current pulse of the third pulse sequence a voltage signal is detected, and additionally no voltage signal is detected upon application of a current pulse having a pulse level corresponding to the second threshold value, the process can be continued using the first or the second pulse sequence.

In all the embodiments of the invention, the current pulses are applied at a frequency of 25 Hz or less, preferably 23 Hz or less, in particular 10 Hz to 15 Hz, particularly preferred 13 Hz.

As explained above, in the processes according to the invention, it has proven to be particularly expedient if the current pulses are applied using a surgical instrument, such as a scalpel, a cannula, or the like. As indicated in the preceding explanation of processes according to the invention, a device for the implementation of such processes has an arrangement for applying current pulses of a specified amplitude, frequency and pulse width in the human or animal body, an electrode arrangement for detecting voltage signals triggered by the current pulses, a device for determining a criterion such as a detection level of voltage signals that are still detectable in response to the current pulses, and a control device for adjusting the amplitude of the current pulses as a function of the voltage signals received in response thereto in such a way that the voltage signals approximately satisfy the criterion, in particular something like the detection level.

The apparatus may have a device for generating optical and/or acoustic warning signals when the amplitude of the current pulses decreases to below a specified threshold value so as to alert the surgeon to an unacceptable approach to a nerve.

In a preferred embodiment of the invention, a carrier frequency amplifier is provided for amplifying the voltage signals, the carrier frequency being specified by the frequency of the current pulses.

As an addition or alternative to a carrier frequency amplifier, a device according to the invention may have a voltage signal detection device in the form of a box car integrator, by means of which a measurement of voltage signals can only take place within a specified, in particular a selectable, window of time after application of the current signal.

In a particularly preferred embodiment of the invention, devices according to the invention have an adjustment facility, across which the surgeon can adjust the location of the operation and the location of the voltage signal detection in such a way that, within the device, the transmission time between application of the current pulses and their detection can be calculated. In this way, the settings of the voltage signal detection device with reference to the window of time, within which the voltage signals are to be detected, can be specified in a simple manner.

Hereinafter, the invention is explained in greater detail with reference to the drawing, to which reference is made relative to all details relevant to the invention and not further highlighted in the description. In the drawing:

### BRIEF DESCRIPTION OF THE DRAWINGS

FIG. 1 shows a schematic representation of a first current pulse sequence,

FIG. 2 shows a schematic representation of a second current pulse sequence, and

FIG. 3 shows a schematic representation of a third current pulse sequence.

### DETAILED DESCRIPTION OF PREFERRED EMBODIMENT

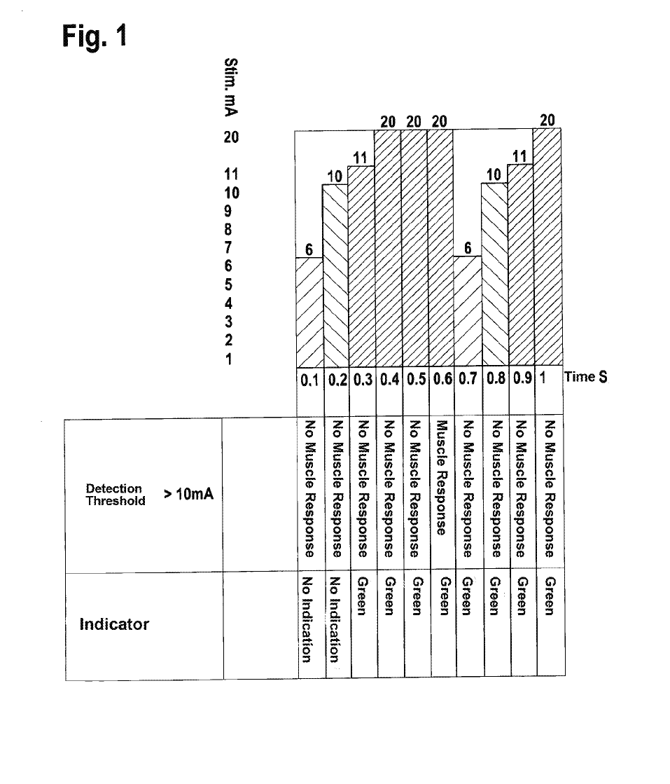

The first current pulse sequence, shown in FIG. 1, comprises a first current pulse having an amplitude of 6 mA corresponding to the second threshold value, a second current pulse having an amplitude of 10 mA corresponding to the first threshold value, a third current pulse having an amplitude of 11 mA, and three successive current pulses having an amplitude of 20 mA each. If none of these current pulses causes a voltage response, it may be safely assumed that the current pulse application is taking place at a sufficient distance from the monitored nerve path. Related thereto, a voltage response will only be accepted if a detection level is reached. In the embodiment of the invention, which is explained based on the drawing, the detection level is determined in that, in a preparatory step, the spontaneous activity of the muscle is first measured without any stimulation using current pulses and the detection level is set to a value exceeding the level of the spontaneous activity by 30 μV to 50 μV.

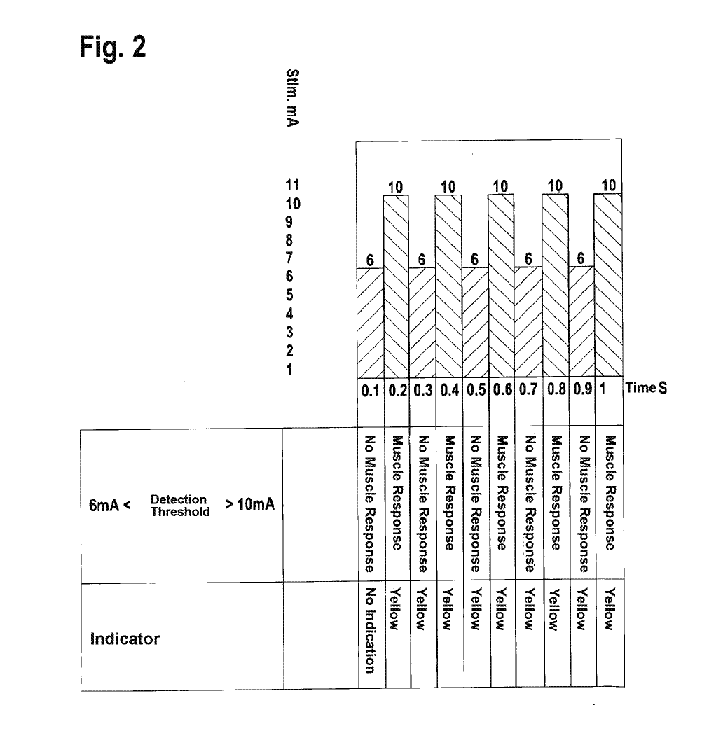

In the second current pulse sequence, shown in FIG. 2, alternating, a first current pulse having an amplitude corresponding to the first threshold value and a second current pulse having an amplitude corresponding to the second threshold value are generated. The first warning signal is triggered if a voltage response can only be detected when the first current pulse is applied.

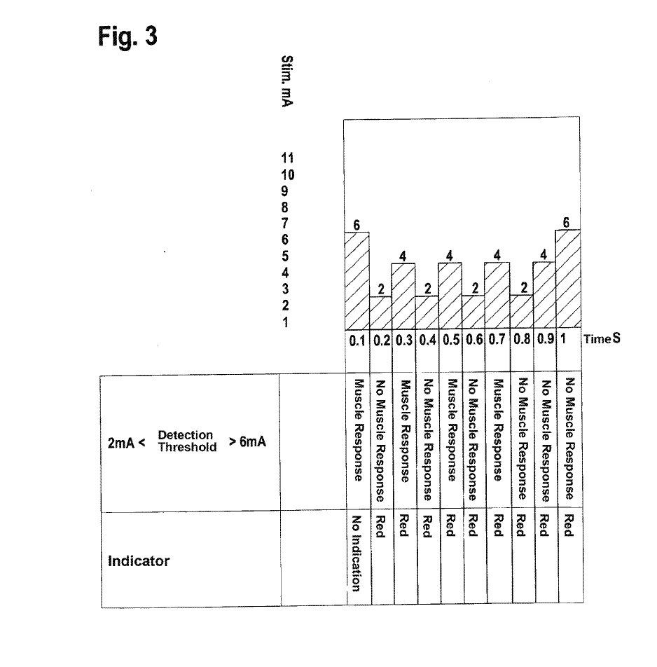

The third current pulse sequence, shown in FIG. 3, is triggered when during the second current pulse sequence, shown in FIG. 2, a voltage response is also obtained in response to a current pulse having an amplitude corresponding to the second threshold value. Then, alternating, current pulses of 2 mA and 4 mA respectively are generated, each having an amplitude below the second threshold value. Only when no voltage signal can be detected in response to any of these pulses having an amplitude of 2 mA and 4 mA, a current pulse having an amplitude of 6 mA corresponding to the second threshold value will be applied again. If even in response to this current pulse no voltage signal can be detected, the application of current pulses according to the second current pulse sequence, illustrated in FIG. 2, takes place automatically. If no signal can be detected even in response to this current pulse sequence, the application of current pulses according to the first current pulse sequence takes place.

1. Process for monitoring a treatment on the human or animal body, in which, in the area of a nerve cord, current pulses of a specified pulse width are applied in the body and, in the area of a muscle in communication with the nerve cord, voltage signals triggered by the current pulses are measured, characterized in that, in a preparatory step, at least one criterion of voltage signals still measurable in response to current pulses is determined and that during the treatment, the amplitude of the current pulses is adjusted in such a way that the voltage signals obtained in response thereto approximately satisfy the criterion.

2. Process according to claim 1, characterized in that a criterion represents a temporal development of the voltage signal.

3. Process according to claim 1, characterized in that the voltage signals are measured using a frequency which is greater than the frequency of the current pulses, in particular at least 10 times the current pulse frequency.

4. Process according to any of the preceding claims, characterized in that one criterion is a detection level of voltage signals that are still measurable in response to current pulses.

5. Process according to claim 4, characterized in that a first warning signal is triggered when the amplitude of the current pulse required for obtaining the detection level decreases to below a first specified threshold value.

6. Process according to claim 4 or claim 5, characterized in that a second warning signal is triggered when the amplitude of the current pulses required for obtaining the detection level decreases to below a second specified threshold value, the second specified threshold level being smaller than the first specified threshold level.

7. Process according to any of the preceding claims 4 to 6, characterized in that for obtaining the detection level, a mean of the voltage signals triggered by the current pulses and the average deviation of the voltage signals from the mean are determined, the detection level being determined as the level, at which the average deviation decreases to below a specified threshold value.

8. Process according to any of the preceding claims, characterized in that for obtaining the detection level, the spontaneous activity of the muscle without any stimulation by current pulses is measured and the detection level is set to a value exceeding the level of the spontaneous activity by a specified rate, preferably 30 μV to 50 μV.

9. Process according to any of the preceding claims 4 to 8, characterized in that the voltage signals are amplified using a carrier frequency amplifier, whose carrier frequency corresponds to the frequency of the current pulses.

10. Process according to any of the preceding claims, characterized in that the current pulses are applied using a surgical instrument, such as a scalpel, a cannula or the like.

11. Process, in particular according to any of the preceding claims, characterized in that a time interval between the application of the current pulses and the measurement of the voltage signals is set.

12. Process according to claim 11, characterized in that the voltage signals are only measured in a specified window of time after application of the current pulses.

13. Process, in particular according to any of the preceding claims, characterized in that, in a first pulse sequence of successive current pulses, a first current pulse with an amplitude having the first specified threshold value, a second current pulse with an amplitude having the second specified threshold value and a third current pulse with an amplitude exceeding the first threshold value are applied and a release signal is generated if none of the current pulses causes a voltage signal which is measurable in response thereto, the first pulse sequence being repeated when the release signal is to be generated.

14. Process according to any of the preceding claims, characterized in that, in a second pulse sequence, alternating, a first current pulse with an amplitude having the first specified threshold value and a second current pulse with an amplitude having the second specified threshold value are generated, the first warning signal being generated and the second pulse sequence being repeated, if only in response to the first current pulse a voltage signal is measurable.

15. Process according to any of the preceding claims, characterized in that a third pulse sequence is generated in response to the detection of a voltage signal as a reaction to a current pulse with an amplitude having the second threshold value, at which (amplitude), alternating, current pulses with different amplitudes, each situated below the second threshold value, being generated, a current pulse with an amplitude corresponding to the second threshold value being then generated again, when in response to no current pulse of the third pulse sequence a voltage signal is detected.

16. Device for implementing a process according to any of the preceding claims.

17. Device according to claim 16 having an arrangement for applying current pulses of a specified amplitude, frequency and pulse width in a human or animal body, an electrode arrangement for detecting voltage signals triggered by the current pulses, a device for determining criteria of the voltage signals, in particular of a detection level of voltage signals still detectable in response to the current pulses and a control device for adjusting the amplitude of the current pulses as a function of the voltage signals obtained in response thereto in such a way that the voltage signals approximately satisfy the criteria.

18. Device according to claim 17, characterized by a device for generating optical and/or acoustic warning signals when the regulated amplitude of the current pulses decreases to below a specified threshold value.

19. Device according to claim 17 or claim 18, characterized by a carrier frequency amplifier for amplifying the voltage signals, the carrier frequency being specified by the frequency of the current pulses.

20. Device according to any of the claims 17 to 19, characterized in that the arrangement for applying the current pulses has a surgical instrument equipped with an electrode arrangement, such as a scalpel, a cannula or the like.

21. Device according to any of the claims 16 to 20 having a voltage signal detection device, by means of which a measurement of voltage signals takes place only within a specified selectable window of time after application of the current signal.
